(19) United States
(12) Patent Application Publication
(10) Pub. No.: US 2025/0176908 A1
(43) Pub. Date: Jun. 5, 2025

(54) 3D LIQUID DIODE AND PERMEABLE ELECTRONIC DEVICES BASED ON THE SAME

(71) Applicant: City University of Hong Kong, Hong Kong (HK)

(72) Inventors: Xinge YU, Hong Kong (HK); Binbin ZHANG, Hong Kong (HK); Jiyu LI, Hong Kong (HK); Jingkun ZHOU, Hong Kong (HK)

(21) Appl. No.: 18/525,903

(22) Filed: Dec. 1, 2023

(51) Int. Cl. A61B 5/00; A41D 1/00; A61B 5/256

(52) U.S. Cl. CPC A61B 5/6804; A41D 1/002; A61B 5/256; A61B 2562/18

(57) ABSTRACT

The present invention provides a three-dimensional liquid diode comprising: a vertical liquid diode layer, a horizontal liquid diode layer disposed above the vertical liquid diode layer, and a liquid collector disposed between the horizontal liquid diode layer and the vertical liquid diode layer. The vertical liquid diode layer comprises: a top surface; a bottom surface; and a plurality of channels, each extending from the top surface to the bottom surface with a vertical hydrophilicity gradient. The horizontal liquid diode layer comprises: an upper surface; a lower surface; a plurality of microstructures distributed on the lower surface with a horizontal structural gradient and configured to couple with the plurality of channels of the vertical liquid diode layer; and a plurality of outlets evenly distributed around the plurality of microstructures, and extending from the upper surface to the lower surface. The liquid collector is coupled to the plurality of outlets.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention generally relates to liquid diode technology. More specifically the present invention relates to a three-dimensional (3D) liquid diode and permeable electronic devices based on the same.

### BACKGROUND OF THE INVENTION

Wearable electronics facilitate noninvasive bio-signal monitoring and environmental conditions sensing, offering significant benefits in personal health management, early disease detection and diagnostics, and sports health. User comfort is a crucial aspect in the design of wearable electronics, influencing their widespread adoption and ongoing commercialization. To achieve this, wearable devices should be thin, soft, lightweight, conformally attached to the skin, and permeable to vapor and sweat. To date, substantial advancements have been made in mechanical and electrical properties. For example, as inspired from nature organisms such as the Nepenthes, liquid diodes have developed to directionally and spontaneously transport fluids, which continuously transports water in a directional manner. However, device permeability remains challenging for real-world applications. Achieving permeability and multifunctionality in a singular, integrated electronic system remains an open question.

### SUMMARY OF THE INVENTION

It is an objective of the present invention to provide an integrated system-level permeable wearable device based on 3D liquid diode, capable of offering a comfortable interface between the skin and the device, ensuring stable signal acquisition and adhesion under sweating conditions, thus allowing for long-term monitoring of biological signals. It is another objective of the present invention to offers a flexible and permeable platform allowing direct and seamless integration of additional functional elements without scarifying perspiration performance, thus can be used to manufacture fully permeable, thin, soft, stretchable, and waterproof wearable electronic devices.

In accordance with a first aspect of the present invention, a three-dimensional liquid diode is provided. The three-dimensional liquid diode comprises: a vertical liquid diode layer, a horizontal liquid diode layer disposed above the vertical liquid diode layer, and a liquid collector disposed between the horizontal liquid diode layer and the vertical liquid diode layer. The vertical liquid diode layer comprises: a top surface; a bottom surface; and a plurality of channels, each extending from the top surface to the bottom surface with a vertical hydrophilicity gradient. The horizontal liquid diode layer comprises: an upper surface; a lower surface; a plurality of microstructures distributed on the lower surface with a horizontal structural gradient and configured to couple with the plurality of channels of the vertical liquid diode layer; and a plurality of outlets evenly distributed around the plurality of microstructures, and extending from the upper surface to the lower surface. The liquid collector is coupled to the plurality of outlets.

In accordance with a second aspect of the present invention, a permeable bio-signal monitoring device is provided. The permeable bio-signal monitoring device comprises a sweat-discharging substrate and a flexible electronic circuit board detachably deposited on the sweat-discharging substrate. The sweat-discharging substrate includes a three-dimensional liquid diode; one or more first magnetic coupling elements integrated in the horizontal liquid diode layer; and one or more permeable electrodes attached on the bottom of the vertical liquid diode layer and configured for receiving bio-signals of a subject wearing the permeable bio-signal monitoring device. The flexible electronic circuit board comprises: one or more second magnetic coupling elements configured to couple to the one or more first magnetic coupling elements respectively; and a microcontroller connected to the permeable electrodes and configured for processing the bio-signals received by the permeable electrodes.

In accordance with a third aspect of the present invention, a textile integrated personal weather station device is provided. The textile integrated personal weather station device comprises a sweat-discharging substrate and a flexible electronic circuit board detachably deposited on the sweat-discharging substrate. The sweat-discharging substrate includes a three-dimensional liquid diode; one or more first magnetic coupling elements integrated in the horizontal liquid diode layer; and a textile integrated with the three-dimensional liquid diode. The flexible electronic circuit board includes: one or more second magnetic coupling elements configured to couple to the one or more first magnetic coupling elements respectively; one or more weather sensors for sensing weather conditions and generating sensing signals; and a wireless communication module for transmitting the sensing signals.

The 3D liquid diode can rapidly self-pump sweat at a rate of 12.6 ml/cm²/min from the skin-device interface to the outlet and prevent backflow, maintaining a comfortable skin-device interface. User study elucidates that this technology exhibits consistent and reliable signal tracking/monitoring, robust and stable adhesive tenacity/strength, and reduced cutaneous discomfort during protracted usage including instances of perspiration, minimized irritation during long-term wear even under sweating conditions. Furthermore, its detachable design based on magnetic coupling effect can enhance reusability of the circuit and reduce costs. Therefore, the present invention provides superior permeability, functionality, scalability, and reconfigurability over current solutions in the field.

### BRIEF DESCRIPTION OF THE DRAWINGS

Embodiments of the invention are described in more details hereinafter with reference to the drawings, in which:

FIGS. 1A and 1B respectively show exploded view and cross-sectional view of a 3D liquid diode according to one embodiment of the present invention.

FIG. 2 shows hydrophilicity gradient in a channel of a vertical liquid diode layer according to one embodiment of the present invention.

FIG. 3 shows structural gradient in microstructures on a horizontal liquid diode layer according to one embodiment of the present invention.

FIG. 4 shows sweat transport mechanism in a 3D liquid diode according to one embodiment of the present invention.

FIG. 5 shows a schematic diagram of a permeable bio-signal monitoring device based on a 3D liquid diode according to one embodiment of the present invention.

FIG. 6 shows an exploded view of a permeable bio-signal monitoring device based on a 3D liquid diode according to one embodiment of the present invention.

FIG. 7A shows an open-mesh permeable electrode and FIG. 7B show a serpentine-shaped electrode unit of the permeable electrode according to one embodiment of the present invention.

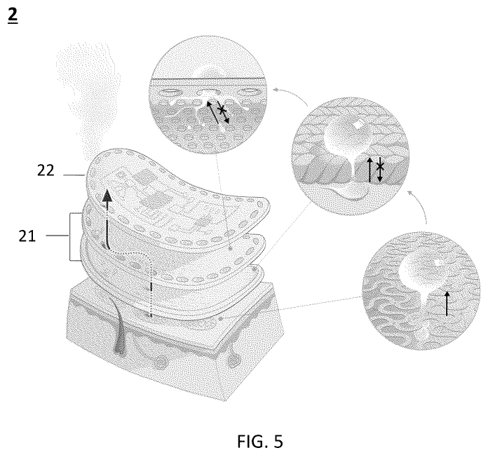

FIG. 5A shows a photo of a prototype of a permeable bio-signal monitoring device installed with a magnetic charging cable according to one embodiment of the present invention; and FIG. 8B shows an exploded view of the magnetic charging cable.

FIG. 9 shows an exploded view of a textile integrated wireless personal weather station device based on a 3D liquid diode according to one embodiment of the present invention.

FIGS. 10A and 10B show fluid dynamics under continuous sweat flow of a PDMS film and a 3D liquid diode respectively.

FIGS. 11A to 11C show the sweat transport and corresponding fluid simulation for the 3D liquid diode undergoing convex bending, concave bending and stretching, respectively.

FIG. 12 shows adhesion strengths of the commercial ECG patch, PDMS film, VLD patch, and 3D liquid diode patch to a user under sweat conditions.

FIG. 13 shows wearing durations of a user wearing the commercial ECG patch, PDMS film, VLD patch, and 3D liquid diode patch until experiencing discomfort at a testing period of 12 hours.

FIG. 14 shows vapor and sweat permeability of the commercial ECG patch, PDMS film, VLD patch, and 3D liquid diode patch.

FIG. 15 shows skin temperature change of a user wearing the commercial ECG patch, PDMS film, VLD patch, and 3D liquid diode patch during physical activity.

FIG. 16 shows comparison of ECG signals acquired using the three types of electrodes: commercial ECG electrodes, PDMS/PI/Au serpentine electrodes, and 3D liquid diode/PI/Au serpentine electrodes from a user doing aerobic exercise.

### DETAILED DESCRIPTION

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a three-dimensional liquid diode is provided to offer a comfortable skin-device interface for long-term monitoring of biological signals and a flexible permeable platform for manufacturing fully permeable, thin, soft, stretchable, and waterproof wearable electronic devices.

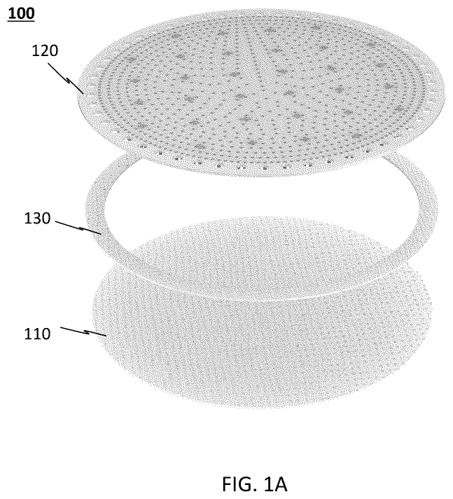

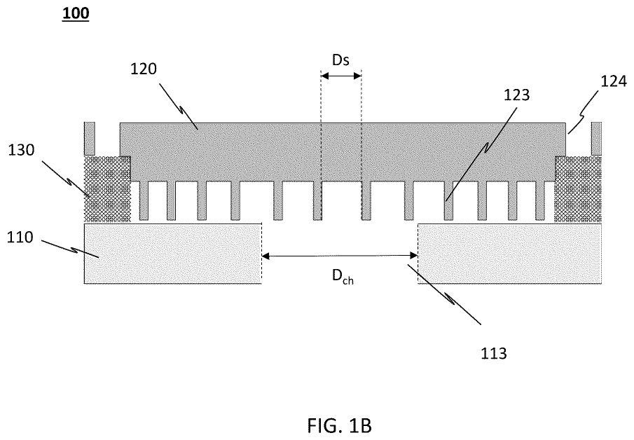

FIGS. 1A and 1B show exploded view and cross-sectional view of a 3D liquid diode 100 according to one embodiment of the present invention. The 3D liquid diode 100 comprises a vertical liquid diode layer 110; a horizontal liquid diode layer 120 disposed above the vertical liquid diode layer 110; and a liquid collector 130 disposed between the vertical liquid diode layer 110 and the horizontal liquid diode layer 120.

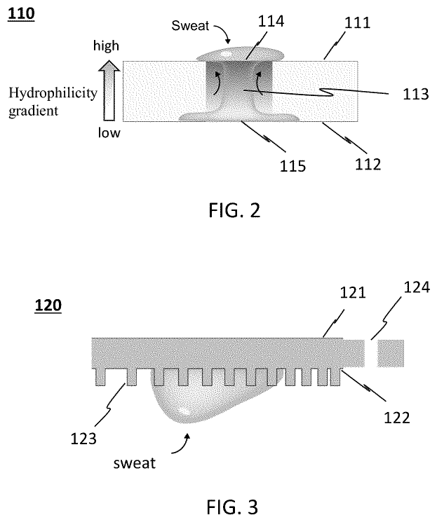

FIG. 2 shows a cross-sectional view of the vertical liquid diode layer 110. The vertical liquid diode layer 110 comprises a top surface 111; a bottom surface 112; and a plurality of channels 113, each extending from the top surface to the bottom surface with a vertical hydrophilicity gradient.

In some embodiments, the vertical hydrophilicity gradient in each channel 113 is configured such that a top end 114 of the channel (at the top surface) is more hydrophilic than a bottom end 115 of the channel (at the bottom surface). As such, sweat can be self-pumped and directed upward through the channel from the bottom surface to the top surface.

In some embodiments, the vertical liquid diode layer is made of a hydrophilic polyester fabric treated with 1H, 1H, 2H, 2H-perfluorooctyltriethoxysilane (PFOTES) and P25 TiO₂ nanoparticles. And the hydrophilicity gradient is formed by a selective oxygen plasma-treatment.

The vertical liquid diode layer may be fabricated by: laser-structuring a polyester fabric (150 μm thick) into specific shapes; dispersing 1H, 1H, 2H, 2H-perfluorooctyltriethoxysilane (PFOTES) (4 ml), P25 TiO₂ nanoparticles (10 g) and deionized (DI) water (1 ml) into ethanol (200 ml) via stirring (for 30 min) to prepare a superhydrophobic suspension; immersing (for 2 hours) the porous fabric in the superhydrophobic suspension to obtain a superhydrophobic porous fabric; dip-coating a single surface (e.g., bottom surface) of the superhydrophobic porous fabric in a water-based acrylic pressure-sensitive adhesive (PSA) to form a permeable adhesive network; applying a silicone PET release film on the permeable adhesive network to protect the PSA, attaching a mask (e.g., made of 50 μm thick laser-structured polyimide (PI) tape) on the non-PSA-coated surface; and performing a selective oxygen plasma-treatment (e.g. PlasmaTherm 790, operated at 200 W, 200 mTorr, O₂ flow rate of 50 sccm) to the masked non-PSA-coated surface to create a hydrophilicity gradient along each microchannel.

FIG. 3 shows a cross-sectional partial view of the horizontal liquid diode layer 120. The horizontal liquid diode layer 120 comprises: an upper surface 121; a lower surface 122; a plurality of super-hydrophilic microstructures 123 distributed with a horizontal structural gradient on the lower surface; and a plurality of outlets 124 surrounding the plurality of microstructures and extending from the upper surface to the lower surface.

In some embodiments, the microstructures are shaped as micropillars and the structural gradient of the distribution of the micropillars is an outward negative gradient of spacing distances Dₛ between adjacent micropillars. That is, micropillars in a central region of the horizontal liquid diode have greater spacing distances than micropillars in a peripheral region which is closer to the outlets. As such, sweat transported from the vertical liquid diode layer can be directed to the outlets.

The vertical liquid diode layer may have a thickness in a range from 125 to 175 μm, preferably equal to 150 μm. The channels may have a diameter substantially equal to 1 mm.

The horizontal liquid diode layer may have a thickness in a range from 450 to 550 μm, preferably equal to 500 μm. The microstructures may have a feature size substantially equal to 50 μm. The spacing distances Ds_x between adjacent microstructures on a x-direction may be given by Ds_x (n)=Ds_x (n−1)+5 μm, where n is a positive integer and Ds_x (1)=40 μm; and the spacing distances Ds_y between adjacent microstructures on a y-direction may be in a range from 50 to 250 μm.

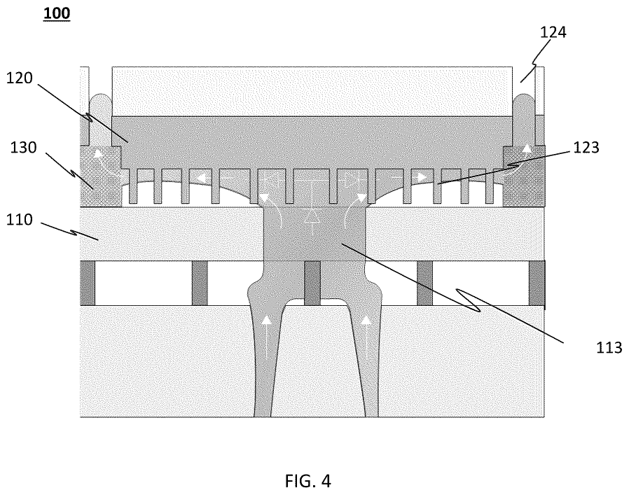

Referring to FIG. 4, the plurality of super-hydrophilic microstructures is configured to couple with the plurality of channels of the vertical liquid diode layer and the liquid collector is configured to couple with the plurality of outlets such that when the 3D liquid diode is attached on skin of a subject, sweat can be self-pumped from eccrine ducts of the subject and vertically drawn up through the channels of the vertical liquid diode layer, then horizontally directed through the microstructures of the horizontal liquid diode layer to the liquid collector, and finally released/discharged through the outlets.

In some embodiments, the horizontal liquid diode layer may be made of polydimethylsiloxane (PDMS, 10:1 of base to curing agent, Sylgard 184, Dow Corning) coated with a super-hydrophilic material.

The horizontal liquid diode layer may be fabricated by: casting polydimethylsiloxane (PDMS) material to form a PDMS layer having a plurality of microstructures arranged with a structural gradient; punching outlet holes surrounding the plurality of microstructures; performing plasm-treatment to the micro-structured PDMS layer; and spray-coating a superhydrophilic coating solution onto the plasma treated PDMS layer (on a hot plate at 110° C. for 10 min) to form the horizontal liquid diode layer.

The casting of the PDMS layer may include the following steps: spin-casting a 100 μm thick photoresist film (e.g., SU8 2050) on a silicon wafer; soft baking at 65° C. for 3 min and 95° C. for 20 min on a hot plate; patterning the spin-casted photoresist film by exposing the wafer under ultraviolet light through a mask; post-exposure baking the wafer at 65° C. for 5 min and 95° C. for 10 min; immersing the wafer in the developer solution for 10 min; laminating PET films on top of the wafer to form a patterned PET layer; treating the patterned PET layer with Octadecyltrichlorosilane by vapor silanization to form a mold; pouring PDMS material into the mold; degassing the PDMS under vacuum; baking the PDMS at 80° C. for 30 min to form the PDMS layer; and releasing the PDMS layer from the mold.

The hydrophilic coating solution may be prepared by dispersing hydrophilic fumed silica (0.1 g) and sodium dodecyl sulfate (SDS) (0.05 g) into 1 wt % polyvinyl alcohol solution (10 ml) to form a mixture; and sonicating (for 10 min) the mixture to obtain the hydrophilic coating solution.

The liquid collector may be fabricated by cleaning a polyester fabric (150 μm thick) with acetone, isopropyl alcohol, and deionized water and then drying at 50° C. for 2 hours; and laser-structured the cleaned polyester fabric.

According to a second aspect of the present invention, a permeable bio-signal monitoring device based on a 3D liquid diode is provided.

Referring to FIG. 5, the permeable bio-signal monitoring device 2 comprises a vapor/sweat-discharging substrate 21 and a flexible electronic circuit board 22 detachably deposited on the vapor/sweat-discharging substrate 21.

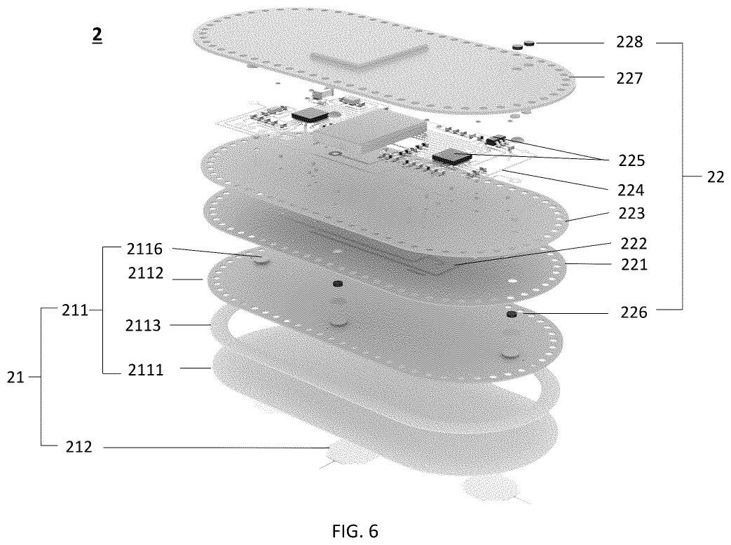

Referring to FIG. 6, the vapor/sweat-discharging substrate 21 may include a 3D liquid diode 211; and one or more permeable electrodes 212 attached on the bottom of the 3D liquid diode and configured for receiving bio-signals (e.g., ECG signals, heart rate (HR), QT interval (QTO), and heart rate variability (HRV)) from skin of a user wearing the monitoring device.

Similar to the 3D liquid diode 100, the 3D liquid diode 211 includes a vertical liquid diode layer 2111; a horizontal liquid diode layer 2112 disposed above the vertical liquid diode layer; and a liquid collector 2113 disposed between the vertical liquid diode layer 2111 and the horizontal liquid diode layer 2112. Additionally, the 3D liquid diode 211 includes one or more magnetic coupling elements 2116 (e.g., iron discs, 2.5 mm diameter and 0.15 mm thickness) integrated into the horizontal liquid diode layer.

The iron discs 2116 may be integrated into the horizontal liquid diode layer 2112 during the PDMS casting process when fabricating the horizontal liquid diode layer. The iron discs may have 2.5 mm diameter and 0.15 mm thickness, and fabricated by laser-patterning (LPKF ProtoLaser U4).

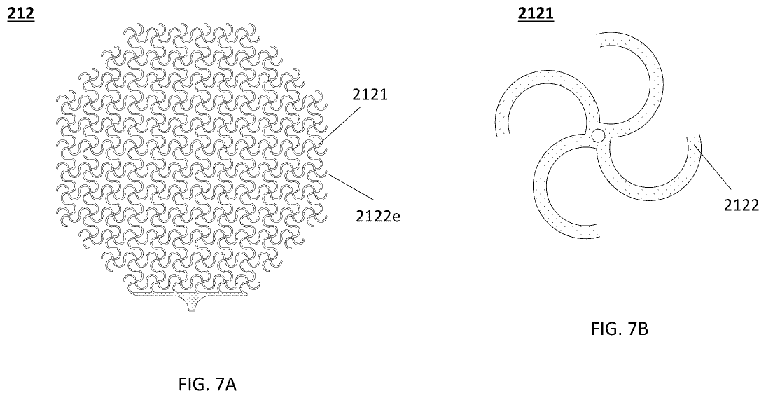

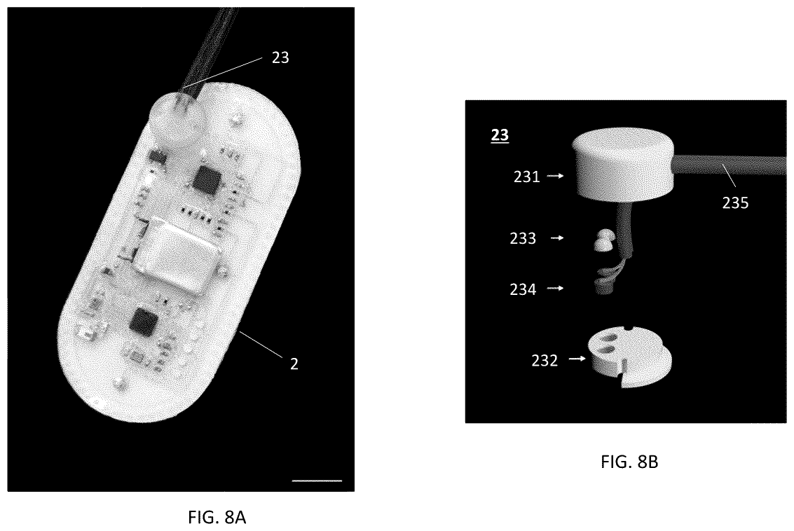

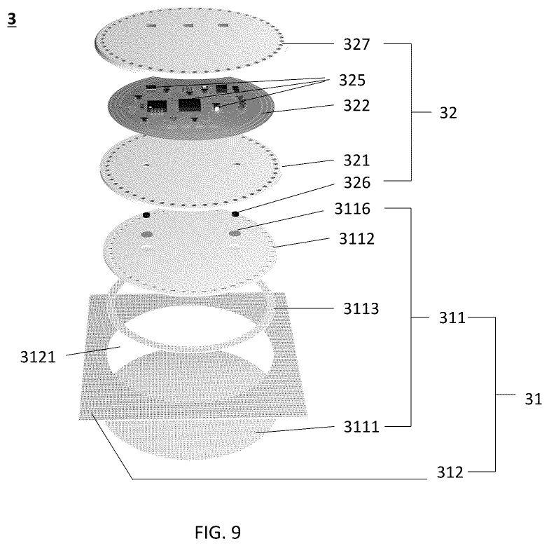

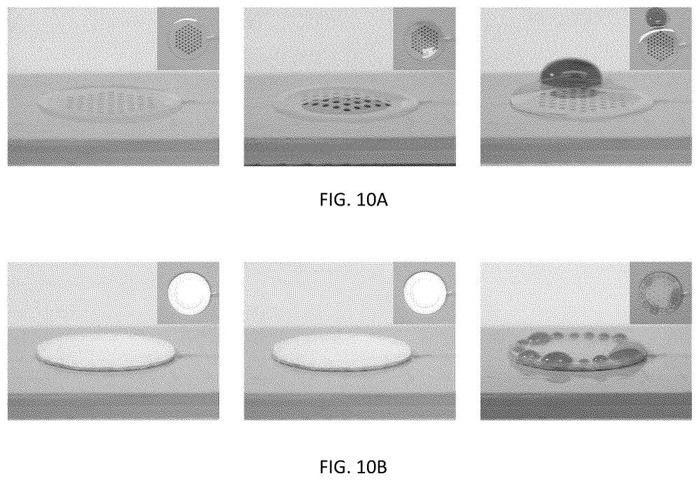

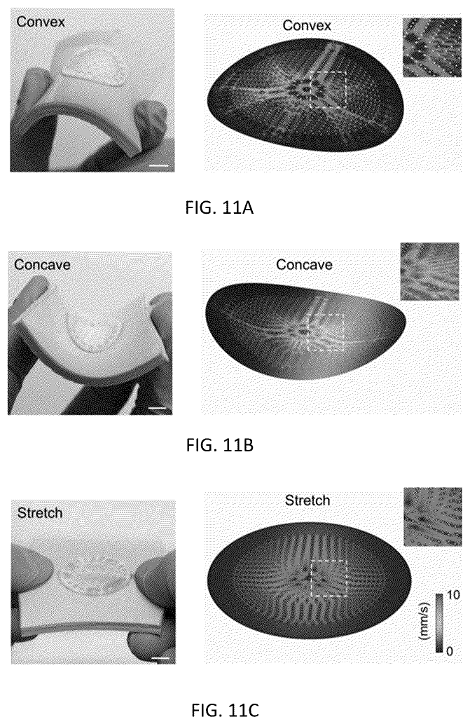

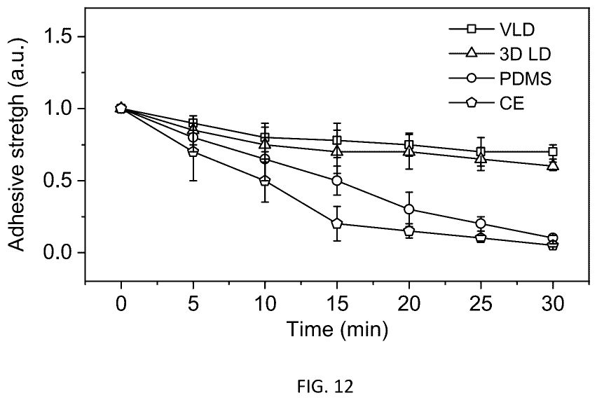

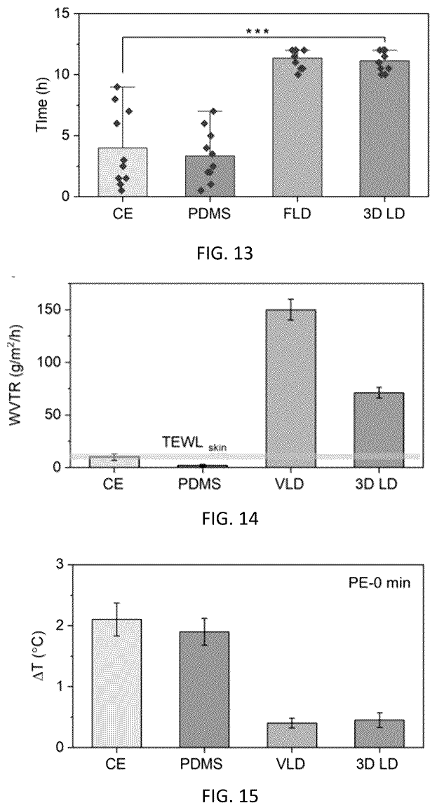

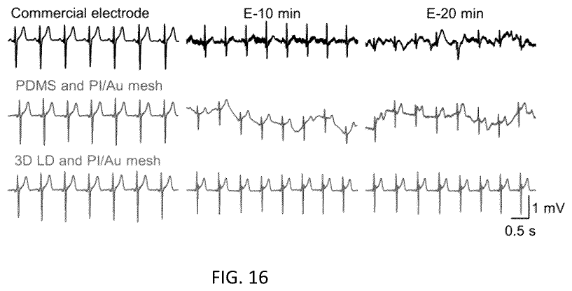

Referring to FIGS. 7A and 7B, each of the permeable electrodes 212 may comprises a plurality of serpentine-shaped PI/Ti/Au electrode units 2121, each having four tails 2122 The plurality of electrode units 2121 is arranged to form an open-mesh electrode network with a plurality of electrode tail ends 2122e situated on the periphery of the network. The PI/Ti/Au open-mesh electrode network may be fabricated by: spin casting PDMS (500 rpm, 30 s) on a glass sheet; baking the spin-casted PDMS (at 100° C. for 10 min); laminating 20-μm thick PI film on the PDMS; patterning the laminated PI film into open mesh architectures by laser cutting (LPKF ProtoLaser U4); using a water-soluble tape (WST) to pick up the patterned PI film from the glass sheet; depositing a Ti/Au (5/50 nm) thin film on the patterned PI film by electron beam evaporation; removing the WST by immersing it into DI water to obtain the Au open-mesh electrode network.

The vapor/sweat-discharging substrate 21 may be assembled by: defining three through holes (0.5 mm diameter) in PDMS portion of the 3D liquid diode by a mechanical punch; passing the electrode tail ends (100-μm width) of the PI/Au open-mesh electrode network through the fabric and through holes and fixing the electrode tails on the iron discs with the silver paste, followed by baking at 80° C. for 30 min; restituting the PDMS pillars (which are punched-out during the mechanical punch) to the through holes and bonding the punched-out PDMS pillars to the through holes with sil-poxy silicone adhesive; attaching PET release film to the bottom of the substrate to protect the PSA adhesive.

Referring back to FIG. 6, the flexible electronic circuit board 22 comprises a flexible substrate 221 with a first patterned interconnection layer 222; a flexible interlayer 223 with a second patterned interconnection layer 224; one or more electrical components 225, magnetic coupling elements 226 and 228 (e.g., magnets), battery; Cu interconnects; and an encapsulation layer 227 covering the electrical components 225.

The one or more electrical components may include a microcontroller connected to the electrode networks and configured for processing the bio-signals received by the electrode networks; a communication module (e.g., a blue-tooth module, or near-field communication (NFC) module); various capacitors and resistances; and a light-emitting diode.

The magnets 226 are positioned such that when the flexible electronic circuit board 22 is deposited on the vapor/sweat-discharging substrate 21, the magnets 226 are coupled to the iron discs 2116 of the vapor/sweat-discharging substrate 21.

The flexible electronic circuit board 22 may be fabricated by: molding a PDMS substrate in a mold; transfer printing a first patterned Cu/PI interconnection layer on the molded PDMS substrate; activating the first patterned Cu/PI interconnection layer and the molded PDMS substrate by ultraviolet ozone for 5 min and heating at 80° C. for 10 min; removing the WST from the first patterned Cu/PI interconnection layer with DI water; placing laser-patterned PET stamps (0.6 mm diameter and 1 mm thickness) with thermal sensitive adhesive on the Cu pads on the first patterned interconnection layer; pouring PDMS into the mold and curing at 40° C. for 12 h to form a PDMS interlayer; removing the PET stamps at 60° C. to form vertical interconnect accesses (VIA) through the PDMS interlayer; transfer printing a second patterned Cu/PI interconnection layer onto the PDMS interlayer in the same way and removing the WST from the second patterned Cu/PI interconnection layer by DI water; soldering the electrical components, magnets, battery and Cu interconnects onto the second patterned Cu/PI interconnection layer with the low melting point solder paste; sealing the circuit board with PDMS and curing at 80° C. for 10 min; removing the circuit board from the mold and laminating the PET release film to the bottom of the circuit board.

Each of the patterned Cu/PI interconnection layers may be fabricated by: laminating a Cu/PI sheet (Cu 18 μm, PI 12.5 μm) on the prepared PDMS/glass substrate; structuring the laminated Cu/PI sheet into a designed pattern by laser cutting (LPKF ProtoLaser U4); picking up the patterned Cu/PI sheet by WST; depositing a Ti/SiO₂ (5/50 nm) thin film on the patterned Cu/PI sheet by electron beam evaporation to form a patterned interconnection layer.

For operation, the magnets in the flexible electronic circuit board and iron discs in the vapor/sweat-discharging substrate can be automatically aligned and mechanically coupled, building the electrical conduction from the electrode networks to the circuit.

Referring to FIGS. 8A and 8B, in some embodiments, the permeable bio-signal monitoring device 2 may further comprise a magnetic charging cable 23 connected to the magnets (228 in FIG. 6). The magnetic charging cable 23 may include a top cover 231, a bottom cover 232, a pair of tin covers 233, a pair of magnets 234, and a USB cable 235. The magnetic charging cable 23 may be fabricated by: fabricating the top and bottom covers by photocuring 3D printing; assembling the USB cable with the top cover; soldering Cu wires in the USB cable on the magnets (1.5 mm diameter, 1 mm thickness) with low melting point solder paste; and installing the bottom cover.

According to a third aspect of the present invention, a textile integrated wireless personal weather station device based on a 3D liquid diode is provided.

Referring to FIG. 9, the textile integrated wireless personal weather station device 3 comprises a textile integrated vapor/sweat-discharging substrate 31 and a flexible electronic circuit board 32 detachably deposited on the textile integrated vapor/sweat-discharging substrate 31.

The textile integrated vapor/sweat-discharging substrate 31 includes a 3D liquid diode 311 and a textile 312 integrated with the 3D liquid diode 311. Similar to the 3D liquid diode 211, the 3D liquid diode 311 includes a vertical liquid diode layer 3111; a horizontal liquid diode layer 3112 disposed above the vertical liquid diode layer 3111; a liquid collector 3113 disposed between the vertical liquid diode layer 3111 and the horizontal liquid diode layer 3112; and one or more magnetic coupling elements 3116 integrated into the horizontal liquid diode layer 3112.

The textile 312 has an opening 3121. The textile 312 is integrated with 3D liquid diode 311 by: placing the textile between the liquid collector 3113 and the vertical liquid diode layer 3111 such that the opening of the textile and the 3D liquid diode are aligned concentrically; and fixing the textile on the vertical liquid diode layer with sil-poxy silicone adhesive.

The flexible electronic circuit board 32 comprises a flexible substrate 321, a flexible printed circuit board 322; and one or more electrical components 325, magnetic coupling elements 326 (e.g., magnets), Cu interconnects; and an encapsulation layer 327 covering the one or more electrical components 325,

The electrical components 325 may include one or more weather sensors for sensing weather conditions and generating sensing signals; and a wireless communication module (e.g., NFC module) for transmitting the sensing signals to a mobile device. For examples, the environmental sensors may include a pressure sensor for sensing atmospheric pressure; an ultra-violet (UV) sensor for sensing intensity of UV lights; a temperature sensor and a humidity sensor.

The flexible electronic circuit board 32 may be fabricated by: soldering the electrical components and magnets (1.5 mm diameter and 0.5 mm thickness) on a flexible printed circuit board (FPCB); placing the FPCB into a 3D printed mold; pouring PDMS with white pigments (silc pig, Smooth On, Inc.) into the mold; degassing and curing the PDMS at 80° C. for 10 min; applying PI shadow masks to insulate the sensors from the PDMS; removing the PI masks after PDMS curing.

### Performance Evaluation

To assess the performance of the 3D liquid diode, a simplified epidermal model is developed to simulate the perspiration process is model incorporates an internal chamber with a perforated surface and is connected to a syringe pump with an adjustable flow rate. For comparison, a PDMS film and the 3D liquid diode are respectively positioned atop the perspiring pores to examine fluid dynamics under continuous sweat flow (FIGS. 10A and 10B).

Due to low gas and water permeability of PDMS film, the continuous liquid injection heightened chamber pressure, leading to the formation of a dome shaped PDMS film structure. In this scenario, further liquid injection caused the fluid to breach the interface between the PDMS and skin model, resulting in a sweat leak and a chamber containing both liquid and gas phases.

Conversely, the 3D liquid diode presents superior breathability and permeability, permitting unhindered transportation of gas and sweat within the internal channel and towards the outlet, thus maintaining conformal contact with the skin model.

Additionally, both experimental and FEA outcomes reveal that the 3D liquid diode sustains stable perspiration transportation capabilities under varied deformations such as convex bending, concave bending and stretching (FIGS. 11A to 11C). Cytotoxicity assessments indicate that the 3D liquid diode exhibits exceptional biocompatibility, with no discernible differences between the samples and the control group.

### User Assessment

A comprehensive user assessment was conducted to investigate the impact of moisture management of a 3D liquid diode based wearable electronic device on both wearable comfort and device performance. The assessment included adhesive tests, evaluations of skin thermal comfort, and assessments of bio-signal quality.

To carry out these evaluations, four different types of patches, including commercial electrocardiogram (ECG) patches, PDMS film, VLD, and 3D liquid diode, are respectively affixed to the upper chest, upper back, and forearm of the subjects. Images of the patch status were captured for investigation while the subjects were engaging in physical activities, specifically playing basketball. Over the 30-minute test period, it was observed that the commercial and PDMS patches detached from the skin successively, while the VLD and 3D liquid diode patches remained firmly attached to the body. It was found that the strength of the patches' adhesion under sweat conditions exhibited a positive correlation with the permeability and softness of the device (FIG. 12).

Furthermore, a long-term wearing test was conducted, in which three groups of samples were affixed to the forearms of three individual subjects respectively for three days of continuous use. No irritation or inflammation of the skin under the VLD and 3D liquid diode patches are observed, whereas the commercial and PDMS patches caused obvious skin erythema.

Another user test was carried out to assess the impact of permeability on wearable comfort. Four types of patches are affixed to the forearms of 10 subjects (5 male and 5 female) and the subjects are instructed to wear the patches for a period of 12 hours. The subjects were free to remove the patches if they experienced any discomfort and then recorded the duration of time they wore each patch.

The results indicated that the 3D liquid diode-based device exhibited longer wearing durations compared with the commercial and PDMS patches (FIG. 13).

The excellent vapor and sweat permeability of 3D liquid diode-based devices contributed to their superior wearable comfort capability (FIG. 14).

Due to the rapid transportation and evaporation of sweat, the 3D liquid diode-based device demonstrated better thermal comfortability during physical activity compared to the commercial and PDMS patches. Infrared images revealed that after running for 15 minutes, the skin temperature rose nearly 2 degrees under the commercial and PDMS patches, while the 3D liquid diode-based devices caused a temperature rise of nearly 0.4 degrees (FIG. 15). The findings suggest that the rapid transportation and evaporation of sweat reduce the thermal accumulation of the skin.

Finally, the effect of sweat accumulation on bio-signal acquisition are evaluated using three types of electrodes: commercial ECG electrodes, PDMS/PI/Au serpentine electrodes, and 3D liquid diode/PI/Au serpentine electrodes (FIG. 16). The electrodes are affixed to the body of a subject in a single-led ECG configuration and tested the ECG signals when the subject exercise for 10 and 20 minutes. With the subject at rest, all electrodes yielded high quality ECG data. However, after 10 minutes of aerobic exercise, the signals obtained from commercially available electrodes and those fabricated from PDMS became unstable and the SNR decreased significantly. The SNR continued to deteriorate, and the ECG signals became progressively more difficult to detect as more perspiration accumulated at the electrode-skin interface. In contrast, the device based on the 3D liquid diode/PI/Au serpentine electrodes featured enhanced moisture permeability and provided stable ECG signals during both the resting and sweating states.

These results suggest permeable electronics based on the 3D liquid diode provided by the present invention are more suitable for continuous and comfortable bio-signal monitoring during strenuous physical activity and in hot ambient conditions that induce perspiration.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A three-dimensional liquid diode, comprising:
a vertical liquid diode layer, comprising:
a top surface;
a bottom surface; and
a plurality of channels, each extending from the top surface to the bottom surface with a vertical hydrophilicity gradient;
a horizontal liquid diode layer disposed above the vertical liquid diode layer, comprising:
an upper surface;
a lower surface;
a plurality of microstructures distributed on the lower surface with a horizontal structural gradient and configured to couple with the plurality of channels of the vertical liquid diode layer; and
a plurality of outlets evenly distributed around the plurality of microstructures, and extending from the upper surface to the lower surface; and
a liquid collector disposed between the horizontal liquid diode layer and the vertical liquid diode layer, and coupled to the plurality of outlets.

2. The three-dimensional liquid diode according to claim 1, wherein the vertical liquid diode layer is made of a hydrophilic polyester fabric.

3. The three-dimensional liquid diode according to claim 2, wherein the hydrophilicity gradient is formed by a selective oxygen plasma-treatment after superhydrophobic treatment with 1H, 1H, 2H, 2H-perfluorooctyltriethoxysilane (PFOTES) and P25 TiO₂ nanoparticles.

4. The three-dimensional liquid diode according to claim 1, wherein the horizontal liquid diode layer is made of polydimethylsiloxane (PDMS) layer coated with a super-hydrophilic material.

5. The three-dimensional liquid diode according to claim 1, wherein the microstructures are micropillars and the structural gradient is a gradient of spacing distances between adjacent micropillars.

6. The three-dimensional liquid diode according to claim 1, wherein the liquid collector is made of a hydrophilic polyester fabric.

7. A permeable bio-signal monitoring device, comprising:
a sweat-discharging substrate including:
a three-dimensional liquid diode of claim 1;
one or more first magnetic coupling elements integrated in the horizontal liquid diode layer; and
one or more permeable electrodes attached on the bottom of the vertical liquid diode layer and configured for receiving bio-signals of a subject wearing the permeable bio-signal monitoring device;
a flexible electronic circuit board detachably deposited on the sweat-discharging substrate and including:
one or more second magnetic coupling elements configured to couple to the one or more first magnetic coupling elements respectively; and
a microcontroller connected to the permeable electrodes and configured for processing the bio-signals received by the permeable electrodes.

8. The permeable bio-signal monitoring device according to claim 7, wherein the vertical liquid diode layer is made of a hydrophilic polyester fabric.

9. The permeable bio-signal monitoring device according to claim 7, wherein the hydrophilicity gradient is formed by a selective oxygen plasma-treatment after superhydrophobic treatment with 1H, 1H, 2H, 2H-perfluorooctyltriethoxysilane (PFOTES) and P25 TiO₂ nanoparticles.

10. The permeable bio-signal monitoring device according to claim 7, wherein the horizontal liquid diode layer is made of a polydimethylsiloxane (PDMS) layer coated with a super-hydrophilic material.

11. The permeable bio-signal monitoring device according to claim 7, wherein the microstructures are micropillars and the structural gradient is a gradient of spacing distances between adjacent micropillars.

12. The permeable bio-signal monitoring device according to claim 7, wherein the liquid collector is made of a hydrophilic polyester fabric.

13. The permeable bio-signal monitoring device according to claim 7, wherein each of the one or more permeable electrodes includes a plurality of serpentine-shaped PI/Ti/Au electrode units arranged to form an open-mesh electrode network.

14. A textile integrated personal weather station device, comprising:
a sweat-discharging substrate including:
a three-dimensional liquid diode of claim 1;
one or more first magnetic coupling elements integrated in the horizontal liquid diode layer; and
a textile integrated with the three-dimensional liquid diode; and
a flexible electronic circuit board detachably deposited on the sweat-discharging substrate and including:
one or more second magnetic coupling elements configured to couple to the one or more first magnetic coupling elements respectively;
one or more weather sensors for sensing weather conditions and generating sensing signals; and
a wireless communication module for transmitting the sensing signals.

15. The textile integrated personal weather station device according to claim 14, wherein the vertical liquid diode layer is made of a hydrophilic polyester fabric.

16. The textile integrated personal weather station device according to claim 14, wherein the hydrophilicity gradient is formed by a selective oxygen plasma-treatment after superhydrophobic treatment with 1H, 1H, 2H, 2H-perfluorooctyltriethoxysilane (PFOTES) and P25 TiO₂ nanoparticles.

17. The textile integrated personal weather station device according to claim 14, wherein the horizontal liquid diode layer is made of a polydimethylsiloxane (PDMS) layer coated with a super-hydrophilic material.

18. The textile integrated personal weather station device according to claim 14, wherein the microstructures are micropillars and the structural gradient is a gradient of spacing distances between adjacent micropillars.

19. The textile integrated personal weather station device according to claim 14, wherein the liquid collector is made of a hydrophilic polyester fabric.

20. The textile integrated personal weather station device according to claim 14, wherein the textile has an opening configured to align to the three-dimensional liquid diode concentrically.
